(19) United States
(12) Patent Application Publication
(10) Pub. No.: US 2022/0022954 A1
(43) Pub. Date: Jan. 27, 2022

(54) ELECTROPORATION CATHETER HAVING TISSUE-CONTACTLESS ELECTRODES

(71) Applicant: Boston Scientific Scimed, Inc., Maple Grove, MN (US)

(72) Inventors: Allan C. Shuros, St Paul, MN (US); Brendan E. Koop, Ham Lake, MN (US)

(21) Appl. No.: 17/383,102

(22) Filed: Jul. 22, 2021

Related U.S. Application Data
(60) Provisional application No. 63/056,298, filed on Jul. 24, 2020.

(51) Int. Cl. A61B 18/14

(52) U.S. Cl. CPC A61B 18/1492; A61B 2018/0022

(57) ABSTRACT

At least some embodiments of the present disclosure are directed to an electroporation ablation catheter having tissue-contactless electrodes. In some embodiments, the electroporation ablation catheter comprises a catheter shaft defining a longitudinal axis and having a proximal end and a distal end; and an electrode assembly extending from the distal end of the catheter shaft, the electrode assembly configured to assume a first collapsed state and a second expanded state. In some cases, the electrode assembly includes an expandable component, and a plurality of electrodes disposed on the expandable component, where in the second state the expandable component have portions configured to protrude from adjacent electrodes.

### CROSS REFERENCE TO RELATED APPLICATION

This application claims priority to Provisional Application No. 63/056,298, filed Jul. 24, 2021, which is herein incorporated by reference in its entirety.

### TECHNICAL FIELD

The present disclosure relates to medical systems and methods for ablating tissue in a patient. More specifically, the present disclosure relates to medical systems and methods for ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells in order to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering. If the electroporation is irreversible, the affected cells are killed through apoptosis.

Irreversible electroporation can be used as a nonthermal ablation technique. In irreversible electroporation, trains of short, high voltage pulses are used to generate electric fields that are strong enough to kill cells through apoptosis. In ablation of cardiac tissue, irreversible electroporation can be a safe and effective alternative to the indiscriminate killing of thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells.

### SUMMARY

As recited in examples, Example 1 is an electroporation ablation catheter. The electroporation ablation catheter comprises a catheter shaft defining a longitudinal axis and having a proximal end and a distal end; and an electrode assembly extending from the distal end of the catheter shaft, the electrode assembly configured to assume a first collapsed state and a second expanded state. The electrode assembly includes an expandable component, and a plurality of electrodes disposed on the expandable component, where the expandable component has a cross-sectional shape defined by a plurality of peaks and a plurality of troughs in the second state, and at least one of the plurality of electrodes is disposed proximate to one of the plurality of troughs.

Example 2 is the electroporation ablation catheter of Example 1, wherein the expandable component comprises a plurality of splines forming a cavity and an inflatable balloon disposed in the cavity, wherein the plurality of splines are generally parallel to the longitudinal axis in the first state and the plurality of splines are expanded outward from the longitudinal axis in the second state, wherein the plurality of electrodes are disposed on or integrated with the plurality of splines, and wherein the balloon is deflated in the first state and the balloon is inflated in the second state, and wherein each one of the plurality of peaks is located between respective adjacent splines, and wherein each one of the plurality of troughs is located proximate one of the plurality of splines.

Example 3 is the electroporation ablation catheter of Example 2, wherein the plurality of splines are mounted to an outer surface of the balloon.

Example 4 is the electroporation ablation catheter of any one of Examples 1-3, wherein one of the plurality of peaks has a first distance from a center point of the cross-sectional shape and one of the plurality of plurality of troughs has a second distance from the center point, and wherein a difference between the first distance and the second distance is in the range of 0.1 millimeters and 5.0 millimeters.

Example 5 is the electroporation ablation catheter of any one of Examples 1-4, wherein the plurality of electrodes comprise a plurality of distal electrodes and a plurality of proximal electrodes, and wherein the plurality of distal electrodes are disposed closer to a distal end of the electroporation ablation catheter than the plurality of proximal electrodes.

Example 6 is the electroporation ablation catheter of Example 2, wherein the balloon is inflated with a fluid.

Example 7 is the electroporation ablation catheter of Example 6, wherein the fluid is a gas.

Example 8 is the electroporation ablation catheter of Example 2, wherein the balloon is semi-complaint.

Example 9 is the electroporation ablation catheter of any one of Examples 1-8, wherein the electroporation ablation catheter is configured to receive an electroporation pulse to the plurality of electrodes and generate an electric field by the plurality of electrodes in the second state.

Example 10 is the electroporation ablation catheter of Example 2, wherein the balloon comprises an insulative material, and wherein the generated electric field is projected outward from an outer surface of the balloon in the second state.

Example 11 is the electroporation ablation catheter of any one of Examples 1-10, wherein at least one of the plurality of electrodes are disposed proximate to one of the plurality of peaks.

Example 12 is the electroporation ablation catheter of Example 2, wherein sections of the balloon are extended radially outward between adjacent splines when inflated.

Example 13 is a system comprising the electroporation ablation device of any one of Examples 1-12.

Example 14 is the system of Example 13, further comprising: a pulse generator configured to generate and deliver ablative energy to the electroporation ablation device.

Example 15 is the system of Example 14, further comprising: a controller coupled to the pulse generator and the electroporation ablation device and configured to control the ablative energy delivered by the pulse generator.

Example 16 is an electroporation ablation catheter. The electroporation ablation catheter comprises a catheter shaft defining a longitudinal axis and having a proximal end and a distal end; and an electrode assembly extending from the distal end of the catheter shaft, the electrode assembly configured to assume a first collapsed state and a second expanded state. The electrode assembly includes an expandable component, and a plurality of electrodes disposed on the expandable component, where in the second state the expandable component has a cross-sectional shape defined by a plurality of peaks and a plurality of troughs, and at least one of the plurality of electrodes is disposed proximate to one of the plurality of troughs.

Example 17 is the electroporation ablation catheter of Example 16, wherein the expandable component comprises a plurality of splines forming a cavity and an inflatable balloon disposed in the cavity, wherein the plurality of splines are generally parallel to the longitudinal axis in the first state and the plurality of splines are expanded outward from the longitudinal axis in the second state, wherein the plurality of electrodes are disposed on or integrated with the plurality of splines, and wherein the balloon is deflated in the first state and the balloon is inflated in the second state, and wherein each one of the plurality of peaks is located between respective adjacent splines, and wherein each one of the plurality of troughs is located proximate one of the plurality of splines.

Example 18 is the electroporation ablation catheter of Example 17, wherein the plurality of splines are mounted to an outer surface of the balloon.

Example 19 is the electroporation ablation catheter of Example 16, wherein one of the plurality of peaks has a first distance from a center point of the cross-sectional shape and one of the plurality of plurality of troughs has a second distance from the center point, and wherein a difference between the first distance and the second distance is in the range of 0.1 millimeters and 5.0 millimeters.

Example 20 is the electroporation ablation catheter of Example 16, wherein the plurality of electrodes comprise a plurality of distal electrodes and a plurality of proximal electrodes, and wherein the plurality of distal electrodes are disposed closer to a distal end of the electroporation ablation catheter than the plurality of proximal electrodes.

Example 21 is the electroporation ablation catheter of Example 17, wherein the balloon is inflated with a fluid.

Example 22 is the electroporation ablation catheter of Example 21, wherein the fluid is a gas.

Example 23 is the electroporation ablation catheter of Example 17, wherein the balloon is semi-complaint.

Example 24 is the electroporation ablation catheter of Example 16, wherein the electroporation ablation catheter is configured to receive an electroporation pulse to the plurality of electrodes and generate an electric field by the plurality of electrodes in the second state.

Example 25 is the electroporation ablation catheter of Example 17, wherein the balloon comprises an insulative material, and wherein the generated electric field is projected outward from an outer surface of the balloon in the second state.

Example 26 is the electroporation ablation catheter of Example 16, wherein at least one of the plurality of electrodes are disposed proximate to one of the plurality of peaks.

Example 27 is a method for electroporation ablations. The method includes the steps of: deploying an electroporation ablation catheter in a first state, the electroporation ablation catheter comprising an expandable component and a plurality of electrodes disposed on the expandable component, wherein the expandable component is collapsed in the first state; disposing the electroporation ablation catheter approximate to a target tissue; operating the electroporation ablation catheter in a second state, wherein the expandable component is expanded in the second state, and wherein the expandable component comprises portions configured to be protruded from adjacent electrodes of the plurality of electrodes in the second state; and generating an electric field at the plurality of electrodes of the catheter, the electric field having an electric field strength sufficient for ablating target tissue via irreversible electroporation.

Example 28 is the method of Example 27, wherein the expandable component comprises a plurality of splines and a balloon disposed within a cavity formed by the plurality of splines, and wherein the plurality of electrodes are disposed on or integrated with the plurality of splines.

Example 29 is the method of Example 28, wherein sections of the balloon are extended radially outward between adjacent splines when inflated

Example 30 is the method of Example 29, wherein the balloon comprises an insulative material, and wherein the generated electric field is projected outward from an outer surface of the balloon in the second state.

Example 31 is an electroporation ablation system. The electroporation ablation system comprises: an electroporation ablation catheter and a controller coupled to the electroporation ablation device and configured to control the electroporation ablation device. The electroporation ablation catheter comprises: a catheter shaft defining a longitudinal axis and having a proximal end and a distal end; and an electrode assembly extending from the distal end of the catheter shaft. The electrode assembly configured to assume a first collapsed state and a second expanded state, the electrode assembly including: an expandable component, and a plurality of electrodes disposed on the expandable component, wherein in the second state the expandable component has a cross-sectional shape defined by a plurality of peaks and a plurality of troughs, and at least one of the plurality of electrodes is disposed proximate to one of the plurality of troughs.

Example 32 is the electroporation ablation system of Example 31, wherein the expandable component comprises a plurality of splines forming a cavity and an inflatable balloon disposed in the cavity, wherein the plurality of splines are generally parallel to the longitudinal axis in the first state and the plurality of splines are expanded outward from the longitudinal axis in the second state, wherein the plurality of electrodes are disposed on or integrated with the plurality of splines, and wherein the balloon is deflated in the first state and the balloon is inflated in the second state, and wherein each one of the plurality of peaks is located between respective adjacent splines, and wherein each one of the plurality of troughs is located proximate one of the plurality of splines.

Example 33 is the electroporation ablation system of Example 32, wherein the plurality of splines are mounted to an outer surface of the balloon.

Example 34 is the electroporation ablation system of Example 31, wherein one of the plurality of peaks has a first distance from a center point of the cross-sectional shape and one of the plurality of plurality of troughs has a second distance from the center point, and wherein a difference between the first distance and the second distance is in the range of 0.1 millimeters and 5.0 millimeters.

Example 35 is the electroporation ablation system of Example 31, wherein the plurality of electrodes comprise a plurality of distal electrodes and a plurality of proximal electrodes, and wherein the plurality of distal electrodes are disposed closer to a distal end of the electroporation ablation catheter than the plurality of proximal electrodes.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 depicts an illustrative system diagram for an electroporation ablation system or device 100, in accordance with embodiments of the subject matter of the disclosure.

FIG. 2A is a diagram illustrating a catheter in an expanded state; FIG. 2B depicts a projected end view of the catheter illustrated in FIG. 2A in the expanded state; and FIG. 2C is a diagram illustrating the catheter illustrated in FIG. 2A in a collapsed state, in accordance with embodiments of the subject matter of the disclosure.

FIG. 3 depicts an illustrative example of electric field generated via a catheter when in operation.

FIG. 4 is an example flow diagram depicting an illustrative method of using an electroporation ablation catheter, in accordance with some embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

As the terms are used herein with respect to measurements (e.g., dimensions, characteristics, attributes, components, etc.), and ranges thereof, of tangible things (e.g., products, inventory, etc.) and/or intangible things (e.g., data, electronic representations of currency, accounts, information, portions of things (e.g., percentages, fractions), calculations, data models, dynamic system models, algorithms, parameters, etc.), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error; differences in measurement and/or manufacturing equipment calibration; human error in reading and/or setting measurements; adjustments made to optimize performance and/or structural parameters in view of other measurements (e.g., measurements associated with other things); particular implementation scenarios; imprecise adjustment and/or manipulation of things, settings, and/or measurements by a person, a computing device, and/or a machine; system tolerances; control loops; machine-learning; foreseeable variations (e.g., statistically insignificant variations, chaotic variations, system and/or model instabilities, etc.); preferences; and/or the like.

Although illustrative methods may be represented by one or more drawings (e.g., flow diagrams, communication flows, etc.), the drawings should not be interpreted as implying any requirement of, or particular order among or between, various steps disclosed herein. However, certain some embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

As used herein, the term “based on” is not meant to be restrictive, but rather indicates that a determination, identification, prediction, calculation, and/or the like, is performed by using, at least, the term following “based on” as an input. For example, predicting an outcome based on a particular piece of information may additionally, or alternatively, base the same determination on another piece of information.

Cryo energy and radio-frequency (RF) energy kill tissues indiscriminately through cell necrosis, which can damage the esophagus, the phrenic nerve, coronary arteries, in addition to other undesired effects. Irreversible electroporation (IRE) uses high voltage, short (e.g., 100 microseconds) pulses to kill cells through apoptosis. IRE can be targeted to kill myocardium, sparing other adjacent tissues including the esophageal vascular smooth muscle and endothelium. The posterior left atrial (LA) wall is embryologically venous tissue and along with the pulmonary veins is high contributor for drivers of atrial tachycardias making it a target for ablation. IRE using unipolar (e.g., catheter tip to cutaneous electrode) configuration generally creates deep lesions but results in extracardiac stimulation of nerves and skeletal muscle. Bipolar configuration reduces this side effect but may have less tissue penetration and be more difficult to achieve transmural lesions. Wide area circumferential ablation using point-by-point RF ablation accomplishes some posterior wall isolation.

There are risks of thermal injury when delivering electroporation ablative energy and arcing due to higher current density at electrode edges. At the same time, experiments show that electroporation may create circumferential and transmural lesions in pectinated tissue of the right atrial appendage. Direct electrode-tissue contact may not be required for the delivery of sufficient electroporation energy to ablate the target tissue. Embodiments of the present disclosure are directed to systems/devices and methods for IRE that are capable of creating transmural lesions while reducing risks of thermal injury. In some embodiments, an exploration ablation catheter including a structure to prevent electrodes from directly contacting tissues is used in such systems and methods. In some embodiments, such structure includes an expandable component having portions configured to be protruded from adjacent electrodes when in operation. In some embodiments, such structure includes an inflatable balloon and a plurality of splines having electrodes disposed thereon, where sections of the balloon are configured to extended radially outward from adjacent splines when inflated.

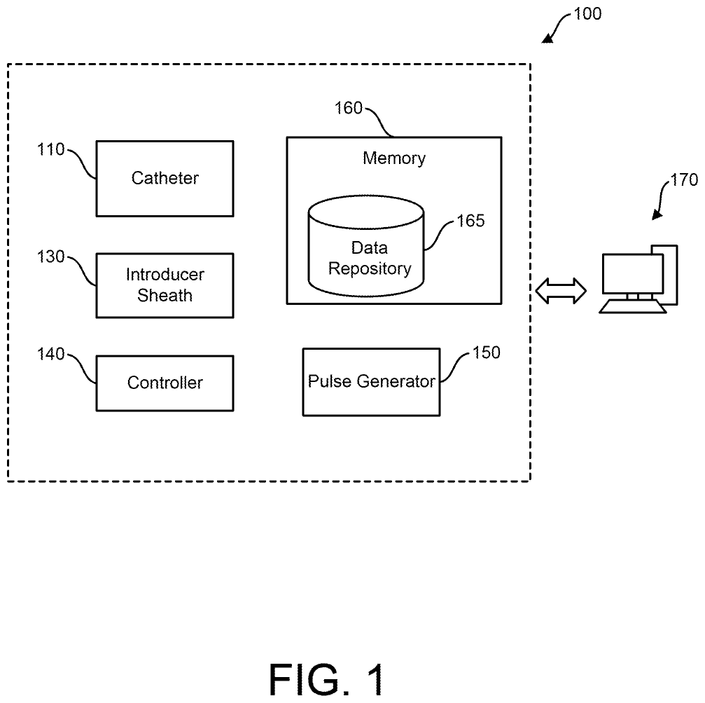

FIG. 1 depicts an illustrative system diagram for an electroporation ablation system or device 100, in accordance with embodiments of the subject matter of the disclosure. The electroporation ablation system/device 100 includes one or more catheters 110, an introducer sheath 130, a controller 140, a pulse generator 150, and a memory 160. In embodiments, the electroporation ablation system/device 100 is configured to deliver electric field energy to targeted tissue in a patient's heart to create tissue apoptosis, rendering the tissue incapable of conducting electrical signals. In some cases, the electroporation ablation system/device 100 may connect with other system(s) 170, for example, a mapping system, an electrophysiology system, and/or the like.

The catheter 110 is designed to be disposed by a target ablation location in the intracardiac chamber. As used herein, an intracardiac chamber refers to cardiac chamber and its surrounding blood vessels (e.g., pulmonary veins). The pulse generator 150 is configured to generate ablative pulse/energy, or referred to as electroporation pulse/energy, to be delivered to electrodes of the catheter 110. The electroporation pulse is typically high voltage and short pulse. The electroporation controller 140 is configured to control functional aspects of the electroporation ablation system/device 100. In embodiments, the electroporation controller 140 is configured to control the pulse generator 150 on the generation and delivery of ablative energy to electrodes of the catheter 110. In one embodiment, the catheter 110 has one or more electrodes. In some cases, each of the one or more electrodes of the catheter 110 is individually addressable. In some cases, the controller 140 may control the ablative energy delivery to each electrode.

In some embodiments, the catheter 110 includes an electrode assembly including one or more electrodes. In some cases, the one or more electrodes are disposed on an expandable component. In some cases, the one or more electrodes are disposed on an outer surface of the expandable component. In some cases, the expandable component comprises portions protruded from adjacent electrodes of the one or more electrodes, when the expandable component is expanded. In such cases, the portions protruded from adjacent electrodes can facilitate contactless operation of the electrodes. In some embodiments, the catheter 110 includes an inflatable balloon and a plurality of splines, where portions of the balloon can extend radially outward (i.e., radially from a longitudinal axis of the catheter) from adjacent splines. In some cases, the one or more electrodes are disposed on or integrated with the plurality of splines, such that outer portions of the balloon (i.e., the portions extended radially outward from adjacent splines) are configured to push tissues away from the electrodes to prevent direct contacts of electrodes with tissues.

In some cases, the electroporation controller 140 receives sensor data collected by sensor(s) of catheter(s) and changes the ablative energy in response to the sensor data. In some cases, the electroporation controller 140 is configured to model the electric fields that can be generated by the catheter 110, which often includes consideration of the physical characteristics of the electroporation catheter 110 including the electrodes and spatial relationships of the electrodes on the electroporation catheter 110. In embodiments, the electroporation controller 140 is configured to control the electric field strength of the electric field formed by the electrodes of the catheter 110 to be no higher than 1500 volts per centimeter. In embodiments, the electroporation catheter 110 allows electrical field to penetrate deeper into the ablation target wall (near-field bipolar) while avoiding skeletal muscle activation that is associated with unipolar (ablation catheter tip to skin electrode).

In embodiments, the electroporation controller 140 includes one or more controllers, microprocessors, and/or computers that execute code out of memory 160, for example, non-transitory machine readable medium, to control and/or perform the functional aspects of the electroporation ablation system/device 100. In embodiments, the memory 160 can be part of the one or more controllers, microprocessors, and/or computers, and/or part of memory capacity accessible through a network, such as the world wide web. In embodiments, the memory 160 comprises a data repository 165, which is configured to store ablation data (e.g., location, energy, etc.), sensed data, modeled electric field data, treatment plan data, and/or the like.

In embodiments, the introducer sheath 130 is operable to provide a delivery conduit through which the electroporation catheter 110 can be deployed to specific target sites within a patient's cardiac chamber. In embodiments, the other systems 170 includes an electro-anatomical mapping (EAM) system. In some cases, the EAM system is operable to track the location of the various functional components of the electroporation ablation system/device 100, and to generate high-fidelity three-dimensional anatomical and electro-anatomical maps of the cardiac chambers of interest. In embodiments, the EAM system can be the RHYTHMIA™ HDx mapping system marketed by Boston Scientific Corporation. Also, in embodiments, the mapping and navigation controller of the EAM system includes one or more controllers, microprocessors, and/or computers that execute code out of memory to control and/or perform functional aspects of the EAM system.

The EAM system generates a localization field, via a field generator, to define a localization volume about the heart, and one or more location sensors or sensing elements on the tracked device(s), e.g., the electroporation catheter pair 105, generate an output that can be processed by a mapping and navigation controller to track the location of the sensor, and consequently, the corresponding device, within the localization volume. In one embodiment, the device tracking is accomplished using magnetic tracking techniques, whereby the field generator is a magnetic field generator that generates a magnetic field defining the localization volume, and the location sensors on the tracked devices are magnetic field sensors.

In some embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements can constitute electrodes on the tracked devices that generate outputs received and processed by the mapping and navigation controller to track the location of the various location sensing electrodes within the localization volume.

In embodiments, the EAM system is equipped for both magnetic and impedance tracking capabilities. In such embodiments, impedance tracking accuracy can, in some instances be enhanced by first creating a map of the electric field induced by the electric field generator within the cardiac chamber of interest using a probe equipped with a magnetic location sensor, as is possible using the aforementioned RHYTHM IA HDx™ mapping system. One exemplary probe is the INTELLAMAP ORION™ mapping catheter marketed by Boston Scientific Corporation.

Regardless of the tracking methodology employed, the EAM system utilizes the location information for the various tracked devices, along with cardiac electrical activity acquired by, for example, the electroporation catheter pair 105 or another catheter or probe equipped with sensing electrodes, to generate, and display via a display, detailed three-dimensional geometric anatomical maps or representations of the cardiac chambers as well as electro-anatomical maps in which cardiac electrical activity of interest is superimposed on the geometric anatomical maps. Furthermore, the EAM system can generate a graphical representation of the various tracked devices within the geometric anatomical map and/or the electro-anatomical map.

Embodiments of the present disclosure allows the electroporation ablation system/device 100 to be used for focal ablations and/or circumference ablations. In some cases, integrated with the EAM system, the system/device 100 allows graphical representations of the electric fields that can be produced by the electroporation catheter pair 105 to be visualized on an anatomical map of the patient and, in some embodiments, on an electro-anatomical map of the patient's heart.

According to embodiments, various components (e.g., the controller 140) of the electroporation ablation system 100 may be implemented on one or more computing devices. A computing device may include any type of computing device suitable for implementing embodiments of the disclosure. Examples of computing devices include specialized computing devices or general-purpose computing devices such “workstations,” “servers,” “laptops,” “desktops,” “tablet computers,” “hand-held devices,” “general-purpose graphics processing units (GPGPUs),” and the like, all of which are contemplated within the scope of FIG. 1 with reference to various components of the system 100.

In some embodiments, a computing device includes a bus that, directly and/or indirectly, couples the following devices: a processor, a memory, an input/output (I/O) port, an I/O component, and a power supply. Any number of additional components, different components, and/or combinations of components may also be included in the computing device. The bus represents what may be one or more buses (such as, for example, an address bus, data bus, or combination thereof). Similarly, in some embodiments, the computing device may include a number of processors, a number of memory components, a number of I/O ports, a number of I/O components, and/or a number of power supplies. Additionally, any number of these components, or combinations thereof, may be distributed and/or duplicated across a number of computing devices.

In some embodiments, the memory 160 includes computer-readable media in the form of volatile and/or nonvolatile memory, transitory and/or non-transitory storage media and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In some embodiments, the memory 160 stores computer-executable instructions for causing a processor (e.g., the controller 140) to implement aspects of embodiments of system components discussed herein and/or to perform aspects of embodiments of methods and procedures discussed herein.

Computer-executable instructions may include, for example, computer code, machine-usable instructions, and the like such as, for example, program components capable of being executed by one or more processors associated with a computing device. Program components may be programmed using any number of different programming environments, including various languages, development kits, frameworks, and/or the like. Some or all of the functionality contemplated herein may also, or alternatively, be implemented in hardware and/or firmware.

The data repository 165 may be implemented using any one of the configurations described below. A data repository may include random access memories, flat files, XML files, and/or one or more database management systems (DBMS) executing on one or more database servers or a data center. A database management system may be a relational (RDBMS), hierarchical (HDBMS), multidimensional (MDBMS), object oriented (ODBMS or OODBMS) or object relational (ORDBMS) database management system, and the like. The data repository may be, for example, a single relational database. In some cases, the data repository may include a plurality of databases that can exchange and aggregate data by data integration process or software application. In an exemplary embodiment, at least part of the data repository 165 may be hosted in a cloud data center. In some cases, a data repository may be hosted on a single computer, a server, a storage device, a cloud server, or the like. In some other cases, a data repository may be hosted on a series of networked computers, servers, or devices. In some cases, a data repository may be hosted on tiers of data storage devices including local, regional, and central.

Various components of the system/device 100 can communicate via or be coupled to via a communication interface, for example, a wired or wireless interface. The communication interface includes, but not limited to, any wired or wireless short-range and long-range communication interfaces. The wired interface can use cables, umbilicals, and the like. The short-range communication interfaces may be, for example, local area network (LAN), interfaces conforming known communications standard, such as Bluetooth® standard, IEEE 802 standards (e.g., IEEE 802.11), a ZigBee® or similar specification, such as those based on the IEEE 802.15.4 standard, or other public or proprietary wireless protocol. The long-range communication interfaces may be, for example, wide area network (WAN), cellular network interfaces, satellite communication interfaces, etc. The communication interface may be either within a private computer network, such as intranet, or on a public computer network, such as the internet.

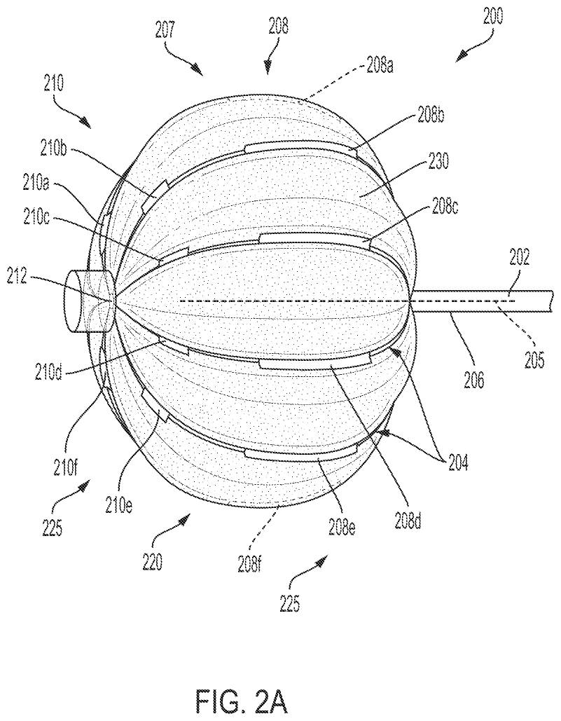

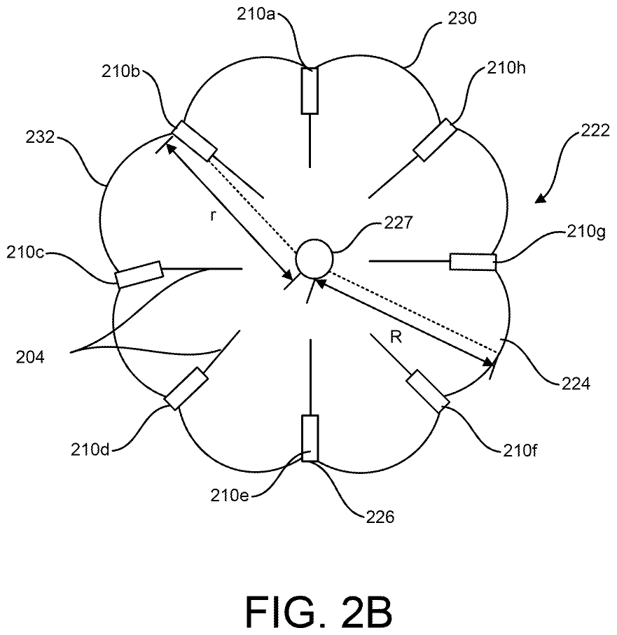

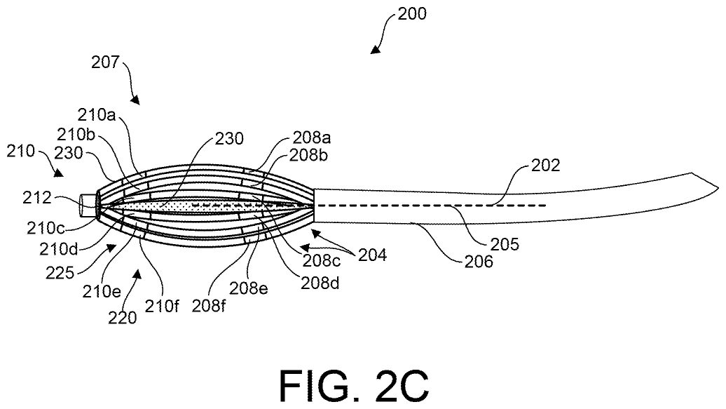

FIG. 2A is a diagram illustrating a catheter 200 in an expanded state; FIG. 2B depicts a projected end view of the catheter 200 in the expanded state; and FIG. 2C is a diagram illustrating the catheter 200 in a collapsed state, in accordance with embodiments of the subject matter of the disclosure. The catheter 200 includes a catheter shaft 202 with a longitudinal axis 205 and having a distal end 206. As used herein, a longitudinal axis refers to a line passing through the centroid of the cross sections of an object. The catheter 200 further includes an electrode assembly 207. In some embodiments, the electrode assembly 207 extends from the distal end 206 of the catheter shaft 202. In embodiments, the electrode assembly 207 is configured to assume a first collapsed state and a second expanded state. In some cases, the electrode assembly 207 includes an expandable component 220 and a plurality of electrodes 225 disposed on the expandable component 220. The expandable component 220 can be collapsed in the first state and expanded in the second state.

In one embodiment, the electrode assembly 207 includes a plurality of splines 204 forming a cavity 215 and an inflatable balloon 230 disposed in the cavity 215. In such embodiment, the plurality of splines 204 and the balloon 230 collectively form the expandable component 220.

In some cases, the plurality of splines 204 are mounted to an outer surface of the balloon 230. In other embodiments, the plurality of splines 204 and the balloon 230 are independent structures, i.e., the splines 204 are not physically attached to the surface of the balloon 230, thus allowing for independent expansion of the splines 204 and the balloon 230.

As illustrated in FIGS. 2A and 2B, when in the second state, the expandable component 220 and/or the inflatable balloon 230 has a cross-sectional shape 222 having peaks 224 and troughs 226. In one embodiment, each one of the peaks 224 is located between respective adjacent splines 204, and wherein each one of the troughs 226 is located proximate one of the plurality of splines 204. In some cases, the expanded component 220 has protruded portions from adjacent electrodes around these peaks 224. In some cases, the balloon 230 has sections extended radially outward from adjacent splines around these peaks 224.

With a non-limiting example illustrated in FIG. 2B, at least one of the plurality of peaks 224 has a first distance R from a center point 227 of the cross-sectional shape 222 and one of the plurality of plurality of troughs 226 has a second distance r from the center point 227. In one embodiment, a difference between the first distance R and the second distance r is in the range of 0.1 millimeters and 5.0 millimeters. In one embodiment, the cross-sectional shape 222 has a plurality of peaks 224 and a plurality of troughs 226. In one example, each of the plurality of peaks has a same distance R to the center point 227. In one example, each of the plurality of troughs has a same distance r to the center point 227.

In one embodiment, the plurality of electrodes 225 are disposed on the outer surface of the expandable component 220. In this embodiment, the expandable component 220 is configured to be protruded (e.g., in areas of 224) from adjacent electrodes of the plurality of electrodes 225 in the second state, for example, to facilitate contactless with tissues. In one embodiment, as illustrated in FIG. 2C, the plurality of splines 204 are generally parallel to the longitudinal axis in the first state. In some embodiments, as illustrated in FIG. 2A, the plurality of splines 204 are expanded outward from the longitudinal axis 205 in the second state, with electrodes 225 disposed on the splines 204. In one example, at least one of the plurality of electrodes 225 is disposed proximate to one of the plurality of peaks 224.

In one embodiments, the inflatable balloon 230 is disposed in the cavity 215, where the balloon 230 is deflated in the first state, with one example illustrated in FIG. 2C; and the balloon 230 is inflated in the second state, with one example illustrated in FIG. 2A. In some cases, the balloon 230 is inflated with a fluid. In some cases, the fluid is saline. In one example, the fluid is a gas. In one example, the fluid is nitrous oxide (N2O). In one case, the balloon 230 is semi-complaint. In another case, the balloon 230 comprises non-complaint material. If the balloon material is non-complaint, the distances from the electrodes to tissues can be known. If the balloon material is semi-complaint, the distances from the electrodes to tissues can be known, for example, with known pressure in the balloon.

In one embodiment, the balloon 230 comprises materials such as, for example, polyvinyl chloride (PVC), polyethylene (PE), cross-linked polyethylene, polyolefins, polyolefin copolymer (POC), polyethylene terephthalate (PET), nylon, polymer blends, polyester, polyimide, polyamides, polyurethane, silicone, polydimethylsiloxane (PDMS) and/or the like. The balloon 230 can comprise relatively inelastic polymers such as PE, POC, PET, polyimide or a nylon material. Membrane 12 can be constructed of relatively compliant, elastomeric materials including, but not limited to, a silicone, latex, urethanes, or Mylar elastomers. The balloon 230 can be embedded with other materials such as, for example, metal, nylon fibers, and/or the like. The balloon 230 can be constructed of a thin, non-extensible polymer film such as, for example, polyester, flexible thermoplastic polymer film, thermosetting polymer film, and/or the like.

In one embodiment, the membrane of the balloon 230 can be about 5-50 micrometers in thickness to provide sufficient burst strength and allow for foldability. In one embodiment, the membrane of the balloon 230 can have a thickness in the range of 25-250 micrometers. In one embodiment, the membrane of the balloon 230 can have tensile strength of 30,000-60,000 psi.

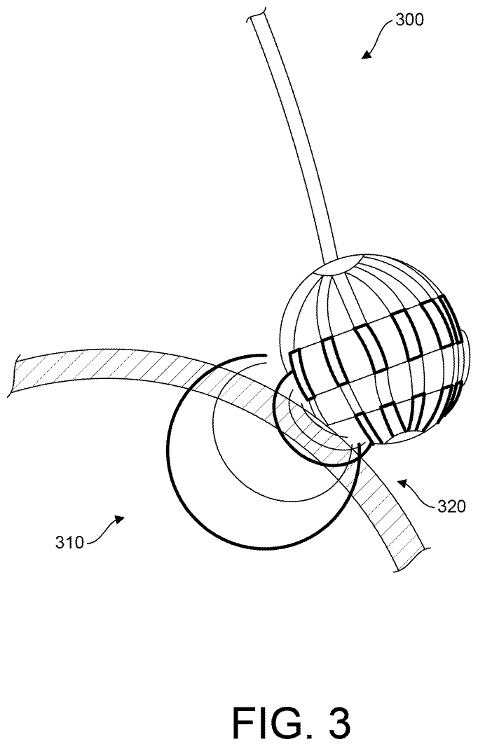

In some embodiments, when in the second state, the electroporation ablation catheter 200 is configured to receive an ablative energy (e.g., electroporation pulse) at the plurality of electrodes 225 and generate an electric field at the electrodes 225. In one embodiment, the electric field has an electric field strength sufficient to ablate a target tissue via irreversible electroporation. In one implementation, the balloon comprises an insulative material, such that the generated electric field is projected outward from the outer surface 232 of the expandable component 220 or the balloon 230. FIG. 3 depicts an illustrative example of electric field 310 generated via a catheter 300 when in operation at a target tissue 320, in accordance with embodiments of the subject matter of the disclosure. As illustrated, the generated electric field 310 is projected outward from the outer surface of the catheter 300 toward the target tissue 320.

In some embodiments, at least some of the electrodes 225 cover 50% or higher surface areas of the respective splines. In some embodiments, at least some of the electrodes 225 cover the entire surface areas of the respective splines. In some embodiments, at least some of the electrodes 225 cover the entire outer surface areas of the respective splines. In some embodiments, the plurality of electrodes 225 includes a first group of electrodes 208 and a second group of electrodes 210. In some cases, the first group of electrodes 208 disposed at the circumference of the plurality of splines 204 and the second group of electrodes 210 disposed adjacent the distal end 212 of the catheter 200. In some cases, the first group of electrodes 208 are referred to as proximal electrodes, and the second group of electrodes 210 are referred to as distal electrodes, where the distal electrodes 210 are disposed closer to the distal end 212 of the electroporation ablation catheter 200 than the proximal electrodes 208. In some implementations, the electrodes 225 can include a thin film of an electro-conductive or optical ink. The ink can be polymer-based. The ink may additionally comprise materials such as carbon and/or graphite in combination with conductive materials. The electrode can include a biocompatible, low resistance metal such as silver, silver flake, gold, and platinum which are additionally radiopaque.

Each of the electrodes in the first group of electrodes 208 and each of the electrodes in the second group of electrodes 210 is configured to conduct electricity and to be operably connected to a controller (e.g., the controller 140 in FIG. 1) and an ablative energy generator (e.g., the pulse generator 150 of FIG. 1). In embodiments, one or more of the electrodes in the first group of electrodes 208 and the second group of electrodes 210 includes flex circuits.

Electrodes in the first group of electrodes 208 are spaced apart from electrodes in the second group of electrodes 210. The first group of electrodes 208 includes electrodes 208a-208f and the second group of electrodes 210 includes electrodes 210a-210f. Also, electrodes in the first group of electrodes 208, such as electrodes 208a-208f, are spaced apart from one another and electrodes in the second of electrodes 210, such as electrodes 210a-210f, are spaced apart from one another.

The spatial relationships and orientation of the electrodes in the first group of electrodes 208 and the spatial relationships and orientation of the electrodes in the second group of electrodes 210 in relation to other electrodes on the same catheter 200 is known or can be determined. In embodiments, the spatial relationships and orientation of the electrodes in the first group of electrodes 208 and the spatial relationships and orientation of the electrodes in the second group of electrodes 210 in relation to other electrodes on the same catheter 200 is constant, once the catheter is deployed.

As to electric fields, in embodiments, each of the electrodes in the first group of electrodes 208 and each of the electrodes in the second group of electrodes 210 can be selected to be an anode or a cathode, such that electric fields can be set up between any two or more of the electrodes in the first and second groups of electrodes 208 and 210. Also, in embodiments, each of the electrodes in the first group of electrodes 208 and each of the electrodes in the second group of electrodes 210 can be selected to be a biphasic pole, such that the electrodes switch or take turns between being an anode and a cathode. Also, in embodiments, groups of the electrodes in the first group of electrodes 208 and groups of the electrodes in the second group of electrodes 210 can be selected to be an anode or a cathode or a biphasic pole, such that electric fields can be set up between any two or more groups of the electrodes in the first and second groups of electrodes 208 and 210.

In embodiments, electrodes in the first group of electrodes 208 and the second group of electrodes 210 can be selected to be biphasic pole electrodes, such that during a pulse train including a biphasic pulse train, the selected electrodes switch or take turns between being an anode and a cathode, and the electrodes are not relegated to monophasic delivery where one is always an anode and another is always a cathode. In some cases, the electrodes in the first and second group of electrodes 208 and 210 can form electric fields with electrode(s) of another catheter. In such cases, the electrodes in the first and second group of electrodes 208 and 210 can be anodes of the fields, or cathodes of the fields.

Further, as described herein, the electrodes are selected to be one of an anode and a cathode, however, it is to be understood without stating it that throughout the present disclosure the electrodes can be selected to be biphasic poles, such that they switch or take turns between being anodes and cathodes. In some cases, one or more of the electrodes in the first group of electrodes 208 are selected to be cathodes and one or more of the electrodes in the second group of electrodes 210 are selected to be anodes. In embodiments, one or more of the electrodes in the first group of electrodes 208 can be selected as a cathode and another one or more of the electrodes in the first group of electrodes 208 can be selected as an anode. In addition, in embodiments, one or more of the electrodes in the second group of electrodes 210 can be selected as a cathode and another one or more of the electrodes in the second group of electrodes 210 can be selected as an anode.

In other embodiments (not shown), a second, outer splined basket assembly can be used in lieu of the balloon 230. That is, the expandable component 220 can be formed of the splines 204, which carry the electroporation electrodes, and a second set of electrically inactive splines interposed between respective splines 204 which, when expanded, extend radially beyond the splines 204 in the same manner as the peaks 224 of the balloon 230. In this manner, the second set of splines provide substantially the same or identical functionality as the balloon 230 described above.

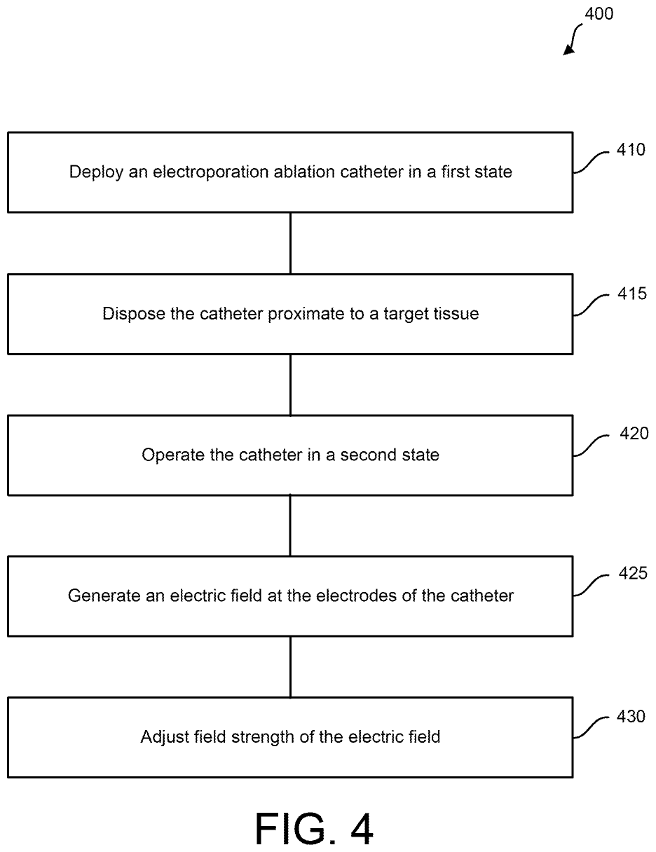

FIG. 4 is an example flow diagram depicting an illustrative method 400 of using an electroporation ablation catheter, in accordance with some embodiments of the present disclosure. Aspects of embodiments of the method 400 may be performed, for example, by an electroporation ablation system/device (e.g., the system/device 100 depicted in FIG. 1). One or more steps of method 400 are optional and/or can be modified by one or more steps of other embodiments described herein. Additionally, one or more steps of other embodiments described herein may be added to the method 400. First, the electroporation ablation system/device deploys an electroporation ablation catheter in a first state (410). In one embodiment, the electroporation ablation catheter includes an expandable component and a plurality of electrodes disposed on the expandable component, where the expandable component is collapsed in the first state.

In embodiments, the electroporation ablation system/device is configured to dispose the electroporation ablation catheter proximate to a target tissue (415). The disposition of the catheter is managed by a controller (e.g., the controller 140 of FIG. 1). The electroporation ablation system/device can operate the catheter in a second state (420), where the expandable component is expanded in the second state, such that the expandable component comprises portions protruded from adjacent electrodes of the plurality of electrodes in the second state. Further, the electroporation ablation system/device generates an electric field at the plurality of electrodes of the catheter (425), where the electric field has an electric field strength sufficient for ablating target tissue via irreversible electroporation. In some cases, the electroporation ablation system/device is configured to deliver exploration pulse to the electrodes.

In some cases, the electroporation ablation system/device is configured to adjust the electric field (430), for example, by changing the exploration pulse and/or the activated electrodes. In one embodiment, the expandable component includes a plurality of splines and a balloon disposed within a cavity formed by the plurality of splines, where the plurality of electrodes are disposed on or integrated with the plurality of splines. In some cases, sections of the balloon are extended radially outward between adjacent splines when inflated. In some designs, the balloon comprises an insulative material, such that the generated electric field is projected outward from an outer surface of the balloon in the second state.

The various embodiments described herein provide significant advantages in irreversible electroporation procedures. The inventors of the present disclosure have determined that intimate tissue-electrode contact is not critical for successful tissue ablation via irreversible electroporation. At the same time, by controllably positioning the ablation electrodes at a known distance away from the target tissue, undesirable physiological effects, e.g., thermal effects resulting from current concentrations at the edges of the ablation electrodes, skeletal muscle capture, and the like, can be greatly minimized or even eliminated altogether.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An electroporation ablation catheter, comprising:
a catheter shaft defining a longitudinal axis and having a proximal end and a distal end; and
an electrode assembly extending from the distal end of the catheter shaft, the electrode assembly configured to assume a first collapsed state and a second expanded state, the electrode assembly including:
an expandable component, and
a plurality of electrodes disposed on the expandable component;
wherein in the second state the expandable component has a cross-sectional shape defined by a plurality of peaks and a plurality of troughs,
wherein at least one of the plurality of electrodes is disposed proximate to one of the plurality of troughs.

2. The electroporation ablation catheter of claim 1, wherein the expandable component comprises a plurality of splines forming a cavity and an inflatable balloon disposed in the cavity, wherein the plurality of splines are generally parallel to the longitudinal axis in the first state and the plurality of splines are expanded outward from the longitudinal axis in the second state, wherein the plurality of electrodes are disposed on or integrated with the plurality of splines, and wherein the balloon is deflated in the first state and the balloon is inflated in the second state, and wherein each one of the plurality of peaks is located between respective adjacent splines, and wherein each one of the plurality of troughs is located proximate one of the plurality of splines.

3. The electroporation ablation catheter of claim 2, wherein the plurality of splines are mounted to an outer surface of the balloon.

4. The electroporation ablation catheter of claim 1, wherein one of the plurality of peaks has a first distance from a center point of the cross-sectional shape and one of the plurality of plurality of troughs has a second distance from the center point, and wherein a difference between the first distance and the second distance is in the range of 0.1 millimeters and 5.0 millimeters.

5. The electroporation ablation catheter of claim 1, wherein the plurality of electrodes comprise a plurality of distal electrodes and a plurality of proximal electrodes, and wherein the plurality of distal electrodes are disposed closer to a distal end of the electroporation ablation catheter than the plurality of proximal electrodes.

6. The electroporation ablation catheter of claim 2, wherein the balloon is inflated with a fluid. The electroporation ablation catheter of claim 6, wherein the fluid is a gas.

8. The electroporation ablation catheter of claim 2, wherein the balloon is semi-complaint.

9. The electroporation ablation catheter of claim 1, wherein the electroporation ablation catheter is configured to receive an electroporation pulse to the plurality of electrodes and generate an electric field by the plurality of electrodes in the second state.

10. The electroporation ablation catheter of claim 2, wherein the balloon comprises an insulative material, and wherein the generated electric field is projected outward from an outer surface of the balloon in the second state.

11. The electroporation ablation catheter of claim 1, wherein at least one of the plurality of electrodes are disposed proximate to one of the plurality of peaks.

12. A method for electroporation ablations, the method comprising:
deploying an electroporation ablation catheter in a first state, the electroporation ablation catheter comprising an expandable component and a plurality of electrodes disposed on the expandable component, wherein the expandable component is collapsed in the first state;
disposing the electroporation ablation catheter approximate to a target tissue;
operating the electroporation ablation catheter in a second state, wherein the expandable component is expanded in the second state, and wherein the expandable component comprises portions configured to be protruded from adjacent electrodes of the plurality of electrodes; and
generating an electric field at the plurality of electrodes of the catheter, the electric field having an electric field strength sufficient for ablating target tissue via irreversible electroporation.

13. The method of claim 12, wherein the expandable component comprises a plurality of splines and a balloon disposed within a cavity formed by the plurality of splines, and wherein the plurality of electrodes are disposed on or integrated with the plurality of splines.

14. The method of claim 13, wherein sections of the balloon are extended radially outward between adjacent splines when inflated

15. The method of claim 12, wherein the balloon comprises an insulative material, and wherein the generated electric field is projected outward from an outer surface of the balloon in the second state.

16. An electroporation ablation system, comprising:
an electroporation ablation catheter comprising:
a catheter shaft defining a longitudinal axis and having a proximal end and a distal end; and
an electrode assembly extending from the distal end of the catheter shaft, the electrode assembly configured to assume a first collapsed state and a second expanded state, the electrode assembly including: an expandable component, and a plurality of electrodes disposed on the expandable component; and
a controller coupled to the electroporation ablation device and configured to control the electroporation ablation device,
wherein in the second state the expandable component has a cross-sectional shape defined by a plurality of peaks and a plurality of troughs,
wherein at least one of the plurality of electrodes is disposed proximate to one of the plurality of troughs.

17. The electroporation ablation system of claim 16, wherein the expandable component comprises a plurality of splines forming a cavity and an inflatable balloon disposed in the cavity, wherein the plurality of splines are generally parallel to the longitudinal axis in the first state and the plurality of splines are expanded outward from the longitudinal axis in the second state, wherein the plurality of electrodes are disposed on or integrated with the plurality of splines, and wherein the balloon is deflated in the first state and the balloon is inflated in the second state, and wherein each one of the plurality of peaks is located between respective adjacent splines, and wherein each one of the plurality of troughs is located proximate one of the plurality of splines.

18. The electroporation ablation system of claim 17, wherein the plurality of splines are mounted to an outer surface of the balloon.

19. The electroporation ablation system of claim 16, wherein one of the plurality of peaks has a first distance from a center point of the cross-sectional shape and one of the plurality of plurality of troughs has a second distance from the center point, and wherein a difference between the first distance and the second distance is in the range of 0.1 millimeters and 5.0 millimeters.

20. The electroporation ablation system of claim 16, wherein the plurality of electrodes comprise a plurality of distal electrodes and a plurality of proximal electrodes, and wherein the plurality of distal electrodes are disposed closer to a distal end of the electroporation ablation catheter than the plurality of proximal electrodes.
